(19) United States
(12) Patent Application Publication
(10) Pub. No.: US 2025/0268270 A1
(43) Pub. Date: Aug. 28, 2025

(54) EDIBLE OIL-IN-WATER NANOEMULSION FORMULATIONS FOR PREHARVEST TREATMENT AND/OR POSTHARVEST PRESERVATION OF FRUITS OR VEGETABLES

(71) Applicant: UNIVERSITAT DE BARCELONA, Barcelona (ES)

(72) Inventors: Paula MUÑOZ ROLDÁN, Barcelona (ES); Tania MESA PARRA, Cornellà de Llobregat (ES); Sergi MUNNÉ BOSCH, Barcelona (ES)

(73) Assignee: UNIVERSITAT DE BARCELONA, Barcelona (ES)

(21) Appl. No.: 18/858,111

(22) PCT Filed: Apr. 18, 2023
(86) PCT No.: PCT/EP2023/059954

§ 371 Date: Oct. 18, 2024

(30) Foreign Application Priority Data
Apr. 22, 2022 (EP) 22382380.8

(51) Int. Cl. A23B 7/154; A23B 7/16

(52) U.S. Cl. CPC A23B 7/154; A23B 7/16

(57) ABSTRACT

They are prepared by a high energy method (e.g. by sonication) from: an oily solution of edible oils (e.g. high-oleic sunflower oil) as solvent, with melatonin (e.g. 100 μM) and edible fat-soluble antioxidant agents (e.g. 100 μM α-tocopherol), and an aqueous solution of edible water-soluble stabilizing agents (e.g. dextrin 1% w/v). These formulations, applied externally (e.g. by spraying) are useful for preharvest application and/or postharvest preservation of fruits or vegetables, particularly of stone fruits, and specifically of sweet cherries. In two varieties of sweet cherries and two varieties of plums, preservations with the formulations are significantly better than those reached with aqueous solutions of melatonin, or with a commercial formulation, or in the absence of treatment.

### CROSS REFERENCE TO RELATED APPLICATIONS

The present application is a U.S. National Phase Application under 35 U.S.C. § 371 of International Patent Application No. PCT/EP2023/059954 filed Apr. 18, 2023, which claims priority of European Patent Application No. 22382380.8 filed April 22. 2022. The entire contents of which are hereby incorporated by reference.

### FIELD OF THE INVENTION

The present invention relates to the technical field of preharvest treatment and/or postharvest preservation of fruits or vegetables by applying chemical formulations.

### BACKGROUND

Fruit and vegetable senescence or decay is an irreversible process in nature that involves a series of changes which are accompanied by a decline in color, flavor and nutrition, and which ultimately shorten shelf life. Known attempts to improve postharvest preservation of fruits or vegetables include physical storage methods (e.g. refrigeration, controlled atmosphere storage, and ventilation), and treatment with some chemicals (e.g. nitric oxide, chlorine dioxide, calcium chloride, methyl salicylate, acetylsalicylic acid, 1-methyl cyclopropane, and β-aminobutyric acid).

Postharvest preservation of several fruits of the genus Prunus, sometimes simply referred to as stone fruits (sweet cherries, plums, peaches, nectarines, apricots, etc.) is very interesting in food industry. Different edible coatings-applied in liquid form by spraying, dipping, brushing, or dripping-have been proposed to improve postharvest preservation of stone fruits.

It is known that application of exogenous melatonin (hormone that is also named N-acetyl-5-methoxytryptamine) improves the postharvest preservation of fruits and vegetables (for a review, cf. e.g.: T. Xu et al.; “Melatonin is a potential target for improving post-harvest preservation of fruits and vegetables”; Frontiers in Plant Science, 2019; doi: 10.3389/fpls.2019.01388). In particular, immersion in aqueous melatonin solutions has been found useful to improve preservation of sweet cherries (cf. e.g.: F. Wang et al.; “Exogenous melatonin delays postharvest fruit senescence and maintains the quality of sweet cherries”; Food Chemistry; 2019; doi.org/10.1016/j.foodchem.2019.125311).

The commercial product Naturcover CP® (Decco Iberica Post Cosecha, S.A.U., Paterna, Valencia, Spain), which contains sucrose, fatty acid esters (E-473) and other additives, is sold for preventing postharvest decay of cherries, plums and other stone fruits (cf. https://www.deccoiberica.es/producto/naturcover-cp/; accessed on 24/01/2022).

Among stone fruits, cherries—particularly sweet cherries (Prunus avium)—are extremely difficult to handle after harvest and are highly perishable, with a shelf life of 7-14 days in cold storage.

Preharvest treatment of fruits and vegetables is also interesting in agriculture, not only for increasing crop yield, but also for improving quality traits at harvest, such as weight, color, firmness, total soluble solids, and titrable acidity. It is known e.g. that foliar spraying with a 0.3 mM aqueous solution of melatonin can be a useful tool to improve crop yield and quality traits of sweet cherries (cf. e.g. A. Carrion-Antoli et al.; “Effects of Melatonin Treatment on Sweet Cherry Tree Yield and Fruit Quality”; Agronomy 2022, vol 12, p. 3. https://doi.org/10.3390/agronomyl2010003; and references therein).

Thus, despite the considerable research and development that has been carried out, there is still room for improvement in the technical field of preharvest treatment and/or postharvest preservation of fruits or vegetables.

### SUMMARY

An aspect of the present invention relates to the provision of an edible oil-in-water (o/w) nanoemulsion formulation prepared by a high energy method involving the use of a mechanical device, from the following: an amount of an oily solution comprising one or more edible oils as solvent, melatonin, and one or more edible fat-soluble antioxidant agents; and an amount of an aqueous solution comprising water as solvent, and one or more edible water-soluble stabilizing agents; wherein the amount of the oily solution to the amount of the aqueous solution is in a v/v ratio between 0.10:100 and 5:100. In particular embodiments, the amount of the oily solution to the amount of the aqueous solution is in a v/v ratio between 1.0:100 and 2.5:100.

In particular embodiments melatonin and the edible fat-soluble antioxidant agents in the oily solution are independently in amounts that yield concentrations between 10 μM and 5.0 mM in the final formulation. In particular embodiments the amounts of melatonin and the edible fat-soluble antioxidant agents are those that independently yield concentrations between 50 μM and 500 μM in the final formulation.

The preparation of the edible o/w nanoemulsion formulations of the present invention can be done from oily solutions having one or more edible oils as solvents. In particular embodiments the edible oils are sunflower oil, which is primarily composed of oleic acid, linoleic acid, palmitic acid and stearic acid, being the four of them in different w/w percentages. In particular embodiments the edible oils are high-oleic sunflower oil, which is stable and has a neutral taste profile, and in which the w/w percentage of oleic acid is in the range 80-90%.

E numbers (“E” stands for “Europe”) are codes for substances used as food additives for use within the European Union and European Free Trade Association. They are commonly found on food labels, and their safety assessment and approval is the responsibility of the European Food Safety Authority. Some food additives with E codes are of natural origin; others are artificially produced; and others have both possibilities.

In particular embodiments the edible fat-soluble antioxidant agents in the oily solution used in the preparation of the edible o/w nanoemulsion formulations are vitamin E vitamers or mixtures thereof. In particular embodiments vitamin E vitamers are tocopherols, which have food additive codes between E306 and E309. In particular embodiments tocopherols are a-tocopherol, which has the food additive code E307.

The edible o/w nanoemulsion formulations of the present invention have oily droplets (typically of diameters <200 nm) dispersed in the aqueous phase. There is no substantial droplet aggregation in the o/w nanoemulsion formulations partially due to the presence of edible water-soluble stabilizing agents in the aqueous solutions that are used to prepare the formulations. In particular embodiments the edible water-soluble stabilizing agents in the aqueous solutions used to prepare the formulations are in w/v concentrations between 0.50% and 5.0% altogether. In particular embodiments they are in w/v concentrations between 1.0% and 2.0% altogether.

In particular embodiments the water-soluble stabilizing agents in the aqueous solution are food additives, particularly: polyvinylpyrrolidone (E1201), poly (vinyl alcohol) (E1203), polydextrose (E1200), starch derivatives (E1401), or mixtures thereof. In particular embodiments, they are starch derivatives (E1401), particularly dextrins (E1400). By the term ‘dextrins’ it is here understood any of the edible members of the group of low-molecular-weight carbohydrates produced by the hydrolysis of starch or glycogen, or mixtures thereof.

In particular embodiments the mechanical device involved in the high energy preparation method is an ultrasonicator, a high pressure valve homogenizer, or a microfluidizer. In particular embodiments the mechanical device is an ultrasonicator.

Another aspect of the present invention relates to the use of the above-disclosed edible o/w nanoemulsion formulations for preharvest treatment and/or postharvest preservation of fruits or vegetables, with the purpose of improving crop yield and/or preservation, respectively. This aspect is closely related to a method of treatment of fruits or vegetables by the application of one or more of the above-disclosed edible o/w nanoemulsion formulations, before or after harvest, with the purpose of improving crop yield and/or preservation. The application of the formulations can be done by spraying, dipping, brushing, or dripping. In particular embodiments the application is done by spraying. In particular embodiments the fruits can be stone fruits. In particular embodiments the stone fruits are sweet cherries.

One of the parameters that is directly related to fruit senescence or decay of fruits is decreasing of firmness index (FI) along storage time (the higher FI, the better for market acceptance). As illustrated by the accompanying comparative tests using sweet cherries and plums (two varieties of each), for the purpose of keeping a high FI of the fruit along storage time, treatments with the edible o/w nanoemulsion formulations of the invention are significantly better than treatments with aqueous solutions of melatonin having the same concentration of this hormone; they are also significantly better than treatments with a commercial formulation sold for preventing postharvest decay of cherries, plums and other stone fruits; and they are significantly better than in the absence of treatment (control).

When fruits and vegetables are infected by pathogens (e.g. fungi), a lack of increase of pathogen infection (PI) along storage time is associated to preservation (the lower the PI, the better for market acceptance). As illustrated by the accompanying comparative tests using sweet cherries and plums (two varieties of each), for the purpose of keeping low PI and particularly when the initial pathogen load is high, treatments with edible o/w nanoemulsion formulations of the invention are significantly better than treatments with aqueous solutions of melatonin having the same concentration of the hormone, and significantly better than in the absence of treatment (control).

Other parameters that are associated to preservation of fruits are: content of total soluble solids (TSS), titrable acidity (TA), and pH. As illustrated by the accompanying comparative tests using sweet cherries and plums (two varieties of each), treatments with edible o/w nanoemulsion formulations of the invention do not significantly change those three parameters, when compared with treatments with aqueous solutions of melatonin having the same concentration of the hormone, treatments with a commercial formulation sold for fruit preservation; and in the absence of treatment (control).

In summary, application of the edible o/w nanoemulsion formulations of the invention to fruits or vegetables, improves their preservation, without being detrimental to their quality.

Throughout the description and claims the word “comprise” and variations of the word, are not intended to exclude other technical features, additives, components, or steps. Furthermore, the word “comprise” encompasses the case of “consisting of”. The following detailed embodiments/examples are provided by way of illustration, and they are not intended to be limiting of the present invention. Additional objects, advantages and features of the invention will become apparent to those skilled in the art upon examination of the description or may be learned by practice of the invention. Furthermore, the present invention covers all possible combinations of particular embodiments described herein.

### DETAILED DESCRIPTION

### Preparation of a Formulation According to the Invention

The following materials were used: α-tocopherol (E-307, Sigma Aldrich, Saint Louis, Missouri, USA), melatonin (Fluka Honeywell Research Chemicals, Seelze, Germany); high oleic sunflower oil (GM Food, Vilamalla, Spain); dextrin (E-1400, Quimics Dalmau, Barcelona, Spain); and deionized and purified water (MiliQ).

A 1.5% w/v aqueous solution of dextrin was prepared by mixing dextrin with water under slow stirring, first at 45° C. for 30 min, and then at room temperature until dextrin was completely dissolved. Separatedly, an oily solution of a-tocopherol and melatonin in high oleic sunflower oil was prepared, having a 667 μM concentration of each product. Then 15 mL of the oily solution was poured into 1000 mL of the aqueous solution of dextrin; thus concentrations of both a-tocopherol and melatonin in the final o/w nanoemulsion formulation were ca. 100 μM. The pouring was done slowly and under stirring. The whole mixture was kept under slow stirring for 10 min; then it was submitted to ultrasonication at 4° C. for 30 min; and finally it was smoothly rotated in a blender (to avoid formation of air bubbles), the o/w nanoemulsion being considered complete when it was transparent or slightly translucent (i.e. when drops were too small to be perceived by the eye). This o/w nanoemulsion formulation is here represented by FINV (from Formulation INVention).

### Preparation of Two Formulations for Comparative Purposes

A formulation of Naturcover CP® (Decco Iberica Post Cosecha, S.A.U., Paterna, Valencia, Spain), obtained by dissolving the commercial product in water following manufacturer recommendations, and here referred to as CNAT (from Comparative NATurcover), was used for comparative purposes. Naturcover CP® is sold for preventing postharvest decay of cherries, plums and other stone fruits.

A 100 μM solution of melatonin in water was also used as a formulation for comparative purposes, as this solution has been suggested for delaying postharvest decay of sweet cherries (cf. e.g. F. Wang et al.; op. cit., p. 2). This formulation here referred to as CMEL (from Comparative MELatonin).

As a negative control, results corresponding to untreated fruits are here represented under CNEG (from Control NEGative).

### Plant Materials and Treatments

Sweet cherries (Prunus avium L.) of two varieties (Prime Giant and Skeena), and plums (Prunus salicina) of two varieties (Angeleno and Rose) were sampled. Fruits were randomly distributed in boxes, having 5 boxes (n=5) for the control (CNEG) and for each of the three formulations (FINV, CNAT and CMEL). Sweet cherries were randomly distributed in the laboratory, and kept in stable conditions of 23° C. and 50% relative humidity (RH). Plums were randomly distributed in the laboratory, and kept in stable conditions of 19° C. and 82% RH.

Each box was respectively sprayed with 20 mL of the three formulations until surfaces of all fruits were completely covered. Samplings and quality parameter determinations were performed at 0, 3 and 5 days for sweet cherries; and at 0, 2, 4 and 8 days for plums, as described below. Values on day 0 correspond to determinations before treatment. Differences in sampling days were due to extension of fruit viability. Pathogen infection (PI) of all fruits was determined every day, from day 0 until the last day of the corresponding analysis period.

Every sampling day, firmness index (FI) was determined first, and then fruits were frozen in liquid nitrogen and stored at −80° C. for determining TSS content, TA and pH (see below). Using the SPSS 25.0 statistical package, all data were statistically processed by two-factor analysis of variance (ANOVA); and two-way multiple comparisons between the control and the four formulations were made.

### Determination of Firmness Index (FI)

FI of fruits was determined with a fruit penetrometer PCE-PTR 200 (PCE Iberica, Albacete, Spain), which measured the force (expressed in Newtons, N) required to push a plunger tip. FI was taken by performing a small cut in the fruit surface and introducing the plunger tip into the fruit flesh until the tip mark. Tips were changed according to fruit type.

### Determination of Pathogen Infection (PI)

PI was determined every day of the experiment. It was expressed as percentage of infected fruits in a box over the total number of fruits in the box. A fruit was considered infected when there were visible symptoms of infection at the fruit surface. PI is believed to be mainly due to fungi.

### Determination of Total Soluble Solids (TSS) Content

TSS content was determined as described (N. Teribia et al.; New Biotechnology; 2016; vol. 33; pp. 824-833), with few modifications. Frozen fruits were grounded until a fine powder was obtained. Samples of 5.0 g of power, kept frozen with liquid nitrogen, were suspended with 50 ml of water. TSS content (expressed in ° Brix) was determined with 1.0 mL of the final sample, using a refractometer (Hanna Instruments, Padova, Italy).

### Determination of Titratable Acidity (TA)

TA was determined as described (N. Teribia et al.; 2016; op. cit.) with few modifications. 10 mL of the final sample were diluted with 100 ml of water, and titrated with 0.10 M NaOH, using 1% phenolphthalein as indicator. TA values are expressed in grams of malic acid by grams of fruit (g.g⁻¹).

### Determination of pH

The pH was determined with a pH-meter in the fruit juice obtained from the frozen material.

### Results of TSS Content, TA, and pH: Statistical Analysis

For the four studied fruit varieties (Prime Giant sweet cherries, Skeena sweet cherries, Angeleno plums, and Rose plums), no statistically significant differences were found in obtained results (not shown) of the three quality parameters in the title, neither versus time, nor versus treatment (CNEG, CNAT, CMEL, and FINV).

### Firmness Index (FI): Results and Conclusions

Obtained FI results for sweet cherries (Prunus avium L.) are shown in Table 1 (Prime Giant) and Table 2 (Skeena), as mean±SD (n=5). Statistically significant differences (P<0.05) between FINV and CNEG, CNAT or CMEL are marked as a, b or c, respectively.

Obtained FI data for plums (Prunus salicina) are shown in Table 3 (Angeleno) and Table 4 (Rose) as mean±SD (n=5). Statistically significant differences between FINV and CNEG, CNAT or CMEL are marked as a, b or c, respectively.

Fruit FI determination during postharvest of sweet cherries and plums showed some statistically significant differences in relation to species and varieties. In all cases fruits treated with the nanoemulsion formulation according to the invention (FINV) kept higher firmness than those in with control/comparative circumstances (CNEG, CNAT, CMEL), and for a longer period of time.

For Prime Giant sweet cherries, after 3 days of postharvest storage, fruits treated with FINV had 22% higher firmness than those untreated (CNEG). Likewise, towards the end of the postharvest storage, 5 days after treatment application, Prime Giant sweet cherries treated with FINV had lost 20% of the initial firmness, but they still had significantly higher firmness (35% more) than those treated with Naturcover® (CNAT, cf. Table 1). A similar pattern was found for Skeena sweet cherries after 5-days of postharvest storage: those treated with FINV had significantly higher firmness than those corresponding to CNEG, CNAT and CMEL (these respectively being 30, 27 and 23% lower, cf. Table 2). In fact, Skeena sweet cherries treated with FINV had constant FI while in the other three cases, FI progressively decreased with time.

FI progression of plums was different from that of cherries. While Angelo plums showed a transient decrease and a final increase (cf. Table 3), Rose plums showed a progressive increase regardless of treatment (cf. Table 4). These results may be related to differences in size and dehydration progression of plums compared with sweet cherries. Nevertheless, at the end of the experiment, 8 days after treatment started, Angeleno plums treated with FINV had better FI (25 and 30%, respectively) than those treated with CNEG and CNAT. At the end of the experiment Rose plums treated with FINV had slightly better FI (only 15%) than those treated with CNEG.

TABLE 1
Firmness index (FI) in Newtons (N), versus time (t)
in days (d), of Prime Giant sweet cherries after
three treatments and a control (no treatment)

| | FI (N) | | | |
|---|---|---|---|---|
| t (d) | CNEG | CNAT | CMEL | FINV |
| 0 | 7.58 ± 0.30 | 7.58 ± 0.30 | 7.58 ± 0.30 | 7.58 ± 0.30 |
| 3 | 6.72 ± 0.42 | 7.34 ± 0.66 | 8.44 ± 0.36 | 8.60 ± 0.36 ^{a} |
| 5 | 4.32 ± 1.03 | 3.96 ± 0.35 | 5.51 ± 0.72 | 6.12 ± 0.89 ^{b} |

TABLE 2
Firmness index (FI) in Newtons (N), versus time (t) in days
(d), of Skeena sweet cherries in four circumstances

| | FI (N) | | | |
|---|---|---|---|---|
| t (d) | CNEG | CNAT | CMEL | FINV |
| 0 | 8.40 ± 0.19 | 8.40 ± 0.19 | 8.40 ± 0.19 | 8.40 ± 0.19 |
| 3 | 7.57 ± 0.45 | 7.10 ± 0.72 | 7.06 ± 0.79 | 6.37 ± 0.45 |
| 5 | 5.32 ± 0.55 | 5.57 ± 0.49 | 5.91 ± 0.38 | 7.64 ± 0.32 ^{a, b, c} |

TABLE 3
Firmness index (FI) in Newtons (N), versus time (t) in days (d), of
Angeleno plums after three treatments and a control (no treatment)

| | FI (N) | | | |
|---|---|---|---|---|
| t (d) | CNEG | CNAT | CMEL | FINV |
| 0 | 33.02 ± 1.22 | 33.02 ± 1.22 | 33.02 ± 1.22 | 33.02 ± 1.22 |
| 2 | 16.05 ± 1.05 | 20.76 ± 1.85 | 17.18 ± 0.62 | 18.77 ± 0.77 |
| 4 | 19.95 ± 1.64 | 15.61 ± 1.52 | 18.05 ± 0.96 | 19.06 ± 1.64 |
| 8 | 24.4 ± 3.0 | 22.9 ± 2.1 | 29.60 ± 1.26 | 32.51 ± 1.14 ^{a, b} |

TABLE 4
Firmness index (FI) in Newtons (N), versus time (t) in days (d),
of Rose plums after three treatments and a control (no treatment)

| | FI (N) | | | |
|---|---|---|---|---|
| t (d) | CNEG | CNAT | CMEL | FINV |
| 0 | 15.14 ± 0.62 | 15.14 ± 0.62 | 15.14 ± 0.62 | 15.14 ± 0.62 |
| 2 | 19.64 ± 0.75 | 20.1 ± 2.2 | 24.22 ± 1.57 | 21.48 ± 0.55 |
| 4 | 21.07 ± 1.07 | 21.32 ± 0.86 | 19.64 ± 0.64 | 21.83 ± 0.63 |
| 8 | 19.43 ± 0.94 | 23.94 ± 0.76 | 22.26 ± 1.22 | 23.05 ± 1.47 ^{a} |

### Patogen Infection (PI): Results and Conclusions

Obtained Pathogen infection (PI) data for sweet cherries (Prunus avium L.) are shown in Table 5 (Prime Giant) and Table 6 (Skeena), as mean±SD (n=5). Statistically significant differences (P<0.05) between FINV and CNEG, CNAT or CMEL are marked as a, b or c, respectively.

Obtained PI data for plums (Prunus salicina) are shown in Table 7 (Angeleno) and Table 8 (Rose) as mean±SD (n=5). Statistically significant differences between FINV and CNEG, CNAT or CMEL are marked as a, b or c, respectively.

Compared with the other three studied fruit varieties, Prime Giant sweet cherries showed the highest PI along all days of the study, for the three treatments and the absence of treatment.

Untreated fruits Prime Giant sweet cherries showed a PI steadily increasing with time, from 0% on day 1, to 85% on day 5 (cf. Table 5, CNEG). For this variety, on a given day no statistically significant differences were found among PI values corresponding to CNEG, CNAT and CMEL treatments. However, on day 5, PI values corresponding to FINV were 25%, 16% and 15% smaller than those corresponding to CNEG, CNAT and CMEL, respectively (cf. Table 5 at t=5 d).

In general, Skeena sweet cherries showed lower PI than Prime Giant sweet cherries, probably due to a difference in the pathogenic load from the orchard. PI of Skeena sweet cherries was clearly visible on day 2, and steadily increased with time (cf. Table 6). On the last day of the study (day 5), PI corresponding to FINV (25%) was not significantly different from PI corresponding to CNAT, but it was significantly smaller than PI corresponding to CNEG and CMEL (ca. 37%).

For Angeleno plums and Rose plums, no statistically significant differences in PI values was observed among the three treatments and the control (cf. Tables 7 and 8). It is noteworthy that Angeleno plums showed very small PI over the 8 days of the study, reaching only a maximum of ca. 15% on day 8. In general, Rose plums showed higher PI than Angeleno plums, similar to those of Skeena sweet cherries, with the particularity that, instead of steadily increasing, they suddenly appeared on day 4 and remained virtually unchanged until day 8.

Thus, an overall conclusion of the study is that FINV treatment is more useful than the other two treatments (CNAT, CMEL) and than the absence of treatment (CNEG), in order to prevent PI in sweet cherries, particularly when the pathogen load is high.

TABLE 5
Patogen infection (PI) in %, versus time (t) in days (d), of Prime Giant
sweet cherries after three treatments and a control (no treatment)

| | PI (%) | | | |
|---|---|---|---|---|
| t (d) | CNEG | CNAT | CMEL | FINV |
| 0 | 0 ± 0 | 0 ± 0 | 0 ± 0 | 0 ± 0 |
| 1 | 13.76 ± 1.93 | 18.2 ± 2.9 | 18.9 ± 4.0 | 10.3 ± 4.3 |
| 2 | 24.9 ± 4.1 | 29.7 ± 5.0 | 34.6 ± 6.8 | 18.1 ± 4.9 |
| 3 | 42.5 ± 7.7 | 44.4 ± 3.9 | 48.1 ± 9.7 | 31.6 ± 6.6 |
| 4 | 69.8 ± 6.3 | 62.0 ± 5.9 | 61.7 ± 9.4 | 44.2 ± 5.5 ^{a, b} |
| 5 | 84.5 ± 3.6 | 81.1 ± 6.3 | 80.1 ± 6.8 | 63.2 ± 4.6 ^{a, b, c} |

TABLE 6
Patogen infection (PI) in %, versus time (t) in days (d), of Skeena
sweet cherries after three treatments and a control (no treatment)

| | FI (N) | | | |
|---|---|---|---|---|
| t (d) | CNEG | CNAT | CMEL | FINV |
| 0 | 0 ± 0 | 0 ± 0 | 0 ± 0 | 0 ± 0 |
| 1 | 0 ± 0 | 0.35 ± 0.03 | 0.75 ± 0.35 | 0 ± 0 |
| 2 | 6.34 ± 1.68 | 4.52 ± 1.62 | 7.92 ± 1.06 | 4.10 ± 1.16 |
| 3 | 9.45 ± 1.11 | 7.28 ± 1.05 | 14.46 ± 1.26 | 8.3± 2.1 |
| 4 | 25.3 ± 2.3 | 20.7 ± 2.8 | 27.2 ± 3.7 | 17.26 ± |
| | | | | 1.49 ^{a, c} |
| 5 | 35.60 ± 1.86 | 27.57 ± 1.31 | 37.2 ± 4.2 | 25.0 ± |
| | | | | 2.9 ^{a, c} |

TABLE 7
Patogen infection (PI) in %, versus time (t) in days (d), of Angeleno
plums after three treatments and a control (no treatment)

| | PI (%) | | | |
|---|---|---|---|---|
| t (d) | CNEG | CNAT | CMEL | FINV |
| 0 | 0.0 ± 0.0 | 0.0 ± 0.0 | 0.0 ± 0.0 | 0.0 ± 0.0 |
| 1 | 0.0 ± 0.0 | 0.0 ± 2.0 | 2.00 ± 0.62 | 0.0 ± 0.0 |
| 2 | 0.0 ± 0.0 | 0.0 ± 2.0 | 2.00 ± 0.62 | 0.0 ± 0.0 |
| 3 | 2.00 ± 0.62 | 2.2 ± 2.2 | 2.2 ± 2.2 | 0.0 ± 0.0 |
| 4 | 4.4 ± 2.7 | 2.2 ± 2.2 | 2.2 ± 2.2 | 0.0 ± 0.0 |
| 5 | 7.5 ± 3.1 | 2.5 ± 2.4 | 2.5 ± 2.4 | 0.0 ± 0.0 |
| 6 | 12.50 ± 1.29 | 2.5 ± 2.4 | 2.5 ± 2.4 | 7.5 ± 5.0 |
| 7 | 12.50 ± 1.29 | 2.5 ± 2.4 | 5.0 ± 3.1 | 7.5 ± 5.0 |
| 8 | 12.50 ± 1.29 | 2.5 ± 2.4 | 5.0 ± 3.1 | 12.5 ± 6.9 |

TABLE 8
Patogen infection (PI) in %, versus time (t) in days (d), of Rose
plums after three treatments and a control (no treatment)

| | FI (N) | | | |
|---|---|---|---|---|
| t (d) | CNEG | CNAT | CMEL | FINV |
| 0 | 0.0 ± 0.0 | 0.0 ± 0.0 | 0.0 ± 0.0 | 0.0 ± 0.0 |
| 1 | 0.0 ± 0.0 | 0.0 ± 0.0 | 1.43 ± 1.43 | 0.0 ± 0.0 |
| 2 | 1.43 ± 1.43 | 5.7 ± 2.7 | 2.9 ± 2.4 | 1.43 ± 1.43 |
| 3 | 3.08 ± 1.88 | 9.2 ± 2.9 | 3.1 ± 2.5 | 18.5 ± 5.2 ^{a, b} |
| 4 | 23.1 ± 4.9 | 24.6 ± 5.1 | 26.2 ± 4.6 | 21.5 ± 5.7 |
| 5 | 26.7 ± 4.9 | 28.3 ± 4.3 | 35.0 ± 3.1 | 23.3 ± 6.1 |
| 6 | 30.0 ± 4.3 | 28.3 ± 3.3 | 35.0 ± 4.1 | 33.3 ± 9.1 |
| 7 | 38.3 ± 4.3 | 35.0 ± 6.7 | 35.0 ± 4.1 | 30.0 ± 9.0 |
| 8 | 36.4 ± 4.1 | 45.5 ± 4.1 | 41.8 ± 2.2 | 41.8 ± 11.7 |

1. An edible oil-in-water nanoemulsion formulation prepared by a high energy method involving the use of a mechanical device, from the following:
an amount of an oily solution comprising:
one or more edible oils as solvent;
melatonin; and
one or more edible fat-soluble antioxidant agents; and
an amount of an aqueous solution comprising:
water as solvent; and
one or more edible water-soluble stabilizing agents;
wherein the amount of the oily solution to the amount of the aqueous solution is in a v/v ratio between 0.10:100 and 5:100.

2. The formulation according to claim 1, wherein the amount of the oily solution to the amount of the aqueous solution is in a v/v ratio between 1.0:100 and 2.5:100.

3. The formulation according to claim 1, wherein melatonin and the edible fat-soluble antioxidant agents in the oily solution are independently in amounts that yield concentrations between 10 μM and 5.0 mM in the final formulation.

4. The formulation according to claim 1, wherein melatonin and the edible fat-soluble antioxidant agents in the oily solution are independently in amounts that yield concentrations between 50 μM and 500 μM in the final formulation.

5. The formulation according to claim 1, wherein the edible oils used as solvent in the oily solution are high-oleic sunflower oil.

6. The formulation according to claim 1, wherein the edible fat-soluble antioxidant agents in the oily solution are vitamin E vitamers or mixtures thereof.

7. The formulation according to claim 6, wherein vitamin E vitamers are α-tocopherol.

8. The formulation according to claim 1, wherein the edible water-soluble stabilizing agents in the aqueous solution are in a w/v concentration between 0.50% and 5.0% altogether.

9. The formulation according to claim 1, wherein the edible water-soluble stabilizing agents in the aqueous solution are in a w/v concentration between 1.0% and 2.0% altogether

10. The formulation according to claim 1, wherein the water-soluble stabilizing agents in the aqueous solution are selected from the group consisting of: polyvinylpyrrolidone, poly(vinyl alcohol), polydextrose, starch derivatives, and mixtures thereof.

11. The formulation according to claim 10, wherein the edible water-soluble stabilizing agents are starch derivatives.

12. The formulation according to claim 11, wherein starch derivatives are dextrins.

13. The formulation according to claim 1, wherein the mechanical device involved in the high energy method is selected from the group consisting of: ultrasonicator, high pressure valve homogenizer, and micro fluidizer.

14. Use of the formulation as defined in claim 1, for preharvest treatment and/or postharvest preservation of fruits or vegetables.

15. The use according to claim 14, wherein fruits are sweet cherries.
